(19) United States
(12) Patent Application Publication
(10) Pub. No.: US 2016/0213072 A1
(43) Pub. Date: Jul. 28, 2016

(54) DEVICES AND METHODS FOR PROTECTING ELDERLY PERSONS

(71) Applicant: Yuk Wan Chung, Hong Kong (HK)

(72) Inventor: Yuk Wan Chung, Hong Kong (HK)

(21) Appl. No.: 14/606,939

(22) Filed: Jan. 27, 2015

(51) Int. Cl. A41D 13/018; A61B 5/11

(52) U.S. Cl. CPC A41D 13/018; A61B 5/1117

(57) ABSTRACT

According to embodiments of the invention, a protective device is used for preventing injuries to an elderly person. In an embodiment, the protective device is a garment assembly having an outer portion defining an inside area and an inner portion defining an outside area. The device is adapted to decide whether a fall has occurred with respect to the elderly person. When a fall is detected, gas is supplied from the inside area to the outside area to inflate the outer portion. Upon inflation, the outer portion provides a soft surface for dampening the impact of the elderly person with a hard surface, thus minimizing the chance of injury.

### FIELD OF THE INVENTION

The invention generally relates to protecting elderly persons from falling. Specifically, this invention relates to protection devices worn by an elderly person to reduce collision impact when falling on the ground.

### BACKGROUND OF THE INVENTION

Serious bodily injuries as a result of accidental falling are a common occurrence with elderly people and in particular, elderly people who have been sick or people who are unsteady on their feet and have difficulty walking. Such accidents occur for example, as a result of slipping on a wet or highly polished floor surface or tripping on objects, for example toys or other objects left on the floor. For elderly people, and in particular those who are sick, injuries causing bone fractures are very difficult to repair and may result in confinement to a wheel chair. Serious bodily injuries causing bone fractures resulting from falling thus represent a serious health concern.

While canes and walkers assist the elderly in being mobile, no devices protect people from the hard impact with a floor or other surface as a result of an accidental fall. As such, there is a need unfulfilled in the art for devices and systems for reducing harm to elderly people and other physically incapacitated people when these people accidentally fall. Therefore, it is an objective of the disclosed technology to provide a safety cushioning system for softening the impact of a person with a hard surface.

### SUMMARY OF THE INVENTION

According to embodiments of the invention, an elderly person may put on a protective device to prevent injuries from falling. The protective device, for example, may be a garment assembly having an outer portion and an inner portion. In one embodiment, an outside area pertaining to an inflatable compartment is substantially within the outer portion of the garment assembly. The inflatable compartment may be made of elastic material that can sufficiently protect the elderly person during operation. Additional features of the garment assembly may include defining an inside area pertaining to a compartment that is substantially within the inner portion of the garment assembly, such that gas can be dispersed into the inside area of the inflatable compartment when the protective device is activated.

In an additional embodiment, a sensor may be included. The sensor may be used to detect motions of the elderly person. There are various motions that may be detected. For example, motions that can be detected by the sensor include detecting orientations, positions, and movements of the garment assembly, such that the elderly person's motions can be captured. In one embodiment, a storage media is included to record the captured information when received from the sensor.

In one other embodiment, a processor is included that performs coordination movement of the protective device. The processor contains a memory instructed to perform an operation of a method. During execution, the embodied method, steps are carried out for preventing the falling and/or injury of the elderly person. There are various steps that can be performed by the processor. An exemplary step includes a step to decide whether a fall has occurred with respect to the elderly person. Additionally, the step includes instructions to supply gas from the inside area to the outside area when the fall occurs, to inflate the elastic material within the outer portion of the garment assembly.

In one embodiment, the step of deciding whether a fall has occurred with respect to the elderly person further entails orientation detection based on the person's previous positions and movements. Additionally, the detection may be performed by the sensor to detect motions of the elderly person. Further, in the process of the detection, a range can be pre-defined so that monitoring can be performed continuously in deciding whether the person is within range of the person's previous normal positions and movements. Even further, in pre-defining the acceptable range of the elderly person's previous normal positions and movements, a threshold value is defined according to an additional embodiment of the present invention, so that the processor can decide if the elderly person's fall has occurred when the person's current positions and movements has reached or exceeded the threshold value.

In one embodiment, the embodied step of deciding whether the fall has occurred further comprises monitor parameters in speed of the fall and rate of change of speed of the fall. The step further comprises monitoring positions including postural orientation of the falling person.

A plurality of steps may involve supplying the gas from the inside area to the outside area. In one example, a valve connecting the inside and outside area of the garment assembly may be triggered such that gas can flow through from the inside area to the outside area. Alternatively, as an additional embodiment, the step of supplying the gas from the inside area to the outside area further entails supplying compressed gas from the inside area to the outside area through the valve. Even further, the step of supplying the gas from the inside area to the outside area further comprises supplying gas from inside area to the outside area through the valve by means of mixing a plurality of chemical compound. Subsequently, after supplying the gas from the inside area to the outside area, the elastic material may be expanded to inflate the outer area to reduce direct physical impact to the person.

According to one embodiment of the present invention, a method is used for preventing injuries to an elderly person. The method entails deciding whether a fall has occurred with respect to the elderly person. When it is detected that a fall has occurred, the gas from the inside area is supplied to the outside area. Many detection parameters can be used. For example, the method includes detecting based on the person's previous positions and movements by the sensor. Also, the method includes deciding whether the person's motion is within range of the person's previous normal positions and movements. Other parameters may relate to speed and rate of the change of the fall. Further, monitored parameters may include elements such as monitoring positions including postural orientation.

In another aspect of the exemplary method, steps may be provided to further supply the gas from the inside area to the outside area when the fall occurs. Specifically, for example, a step may include triggering a valve connecting the inside and outside area of the garment assembly. Further, the inside area may supply gas to the outside area through a valve, which can also be used, alternatively, as a channel to supply gas from inside area to the outside area by means of mixing a plurality of chemical compound, so that the elastic material of the outer area can be expanded to reduce direct physical impact to the person.

In accordance with yet another embodiment, a protective device for preventing injuries to an elderly person is provided. For example, the device may have a garment assembly having an outer portion and an inner portion. The garment assembly can be worn by the elderly person. Additionally, an outside area may contain a plurality of inflatable compartments being substantially within the outer portion of the garment assembly. Each inflatable compartment, in an embodiment, may be made of elastic material, such that each inflatable compartment is isolated from one another. Further, each inflatable compartment can be adapted to inflate selectively to protect different parts of the elderly person during operation. In a different embodiment, an inside area pertaining to a compartment being substantially within the inner portion of the garment assembly is provided. In the embodiment, the inside area disperses gas into each inflatable compartment selectively when the protective device is activated.

It should be understood that the foregoing general description and below detailed description are exemplary and should not be restricted to particular examples, embodiments, or implementations, as claimed.

### BRIEF DESCRIPTION OF THE DRAWINGS

For the purpose of illustrating the bodily protective garment for the elderly, the following drawings show forms which are presently preferred. It is to be understood that this invention is not necessarily limited to the precise arrangement, instrumentalities and field of utility as therein demonstrated.

FIG. 1 shows a protective device worn by an elderly person about to experience a fall, according to an embodiment of the present invention.

FIG. 2 is a cross sectional view of the garment assembly comprised in the protective device exemplary to an aspect of the present invention.

FIG. 3 shows another cross section view of the garment assembly when gas is dispersed from the inside area to the outside area.

FIG. 4 shows another cross section view of the inventive protective device in accordance with another embodiment of the present invention.

FIG. 5 shows another cross section view of the inventive protective device in accordance with another embodiment of the present invention.

FIG. 6 shows an exemplary method to preventing injuries to an elderly person.

FIG. 7 shows an exemplary method of deciding whether a fall has occurred.

FIG. 8 shows an exemplary method of supplying gas from the inside area to the outside area of the inventive protective device.

### DETAILED DESCRIPTION

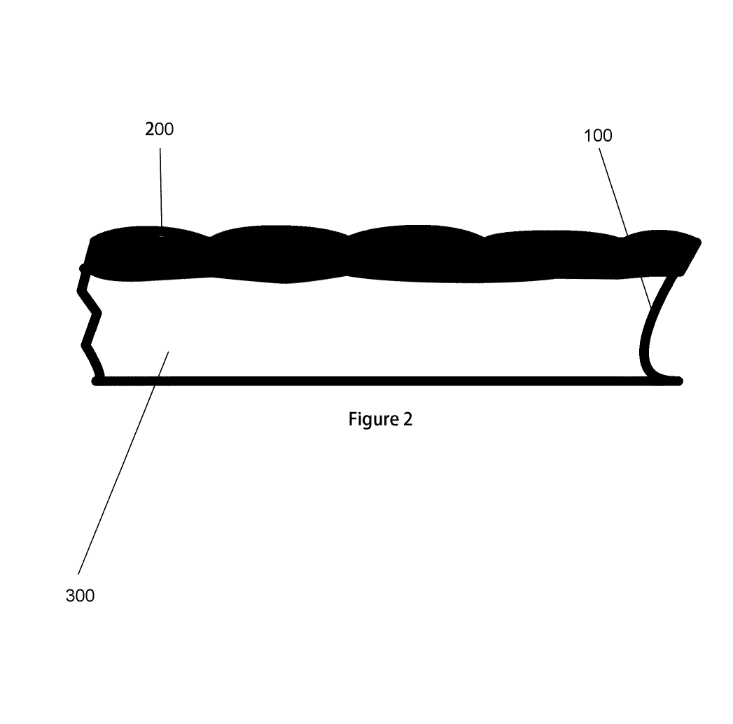

In the embodiments of a protective device to be worn by persons of all ages, engaged in bodily-injury, susceptible activities, an outer inflatable protective device is shown with an inner air-tight, non-inflatable tubing or enclosure. FIGS. 2 to 5 show various constructions or designs of the enclosure to be worn by a person that may give rise to protection to different parts of one's body, such as backs, hips, and knees. In theory, the protective device in the enclosures would protect not only the elderly people but also people of different ages.

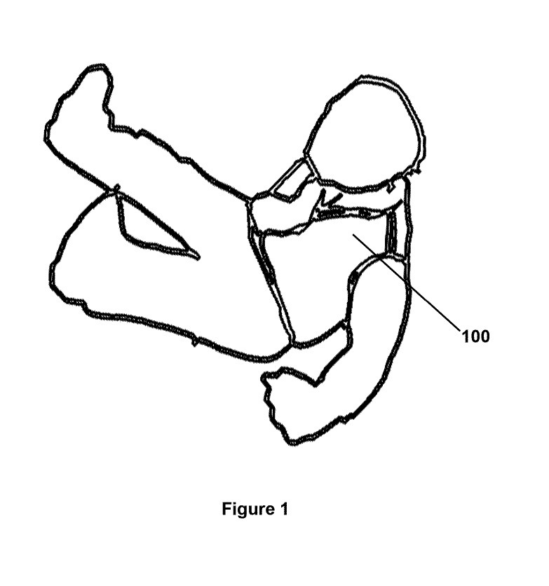

FIG. 1 shows an elderly man wearing a protective device embracing his body. The elderly man is shown experiencing a stumble and in the process of falling down. The protective device comprises a garment assembly 100. When the elderly man is about to fall, a sensor included in the protective device is alerted for the sudden change of orientations, positions, and movements of the garment assembly. The sensor may be a proximity sensor or even a gyroscope that may detect the sudden change of motions. Sending signals from the sensor are passed to a storage media device and a processor unit, which is adapted to further process the received information to decide whether a fall has occurred with respect to the elderly man wearing the protective device. When falling signals are detected, the processor instructs the garment assembly 100 to inflate in order to protect the elderly man.

There are different ways to decide whether a fall has occurred with respect to the elderly person. One example is to use the gyroscope embedded in the protective device. However, to prevent giving rise to false alarms in producing better quality results, the elderly man's habitual positions and movements should be accounted for before deciding to inflate the protective garment. Otherwise, the protective garment would be falsely inflated that may give rise to inconvenience to the elderly man. As an example, the elderly person may simply bend down his knees to pick up certain items on the floor. If the bending movement causes the protective garment to operate, a feeling of awkwardness would result to the elderly person. One resolution to the problem is to record the daily movement of the person wearing the protective wear. That includes the speed and rate of change of speed of movement of the person. When there is a sharp increase of the speed or rate or change of speed of the monitoring movement, comparisons may be made before deciding to actuate the panic button to rescue the falling person.

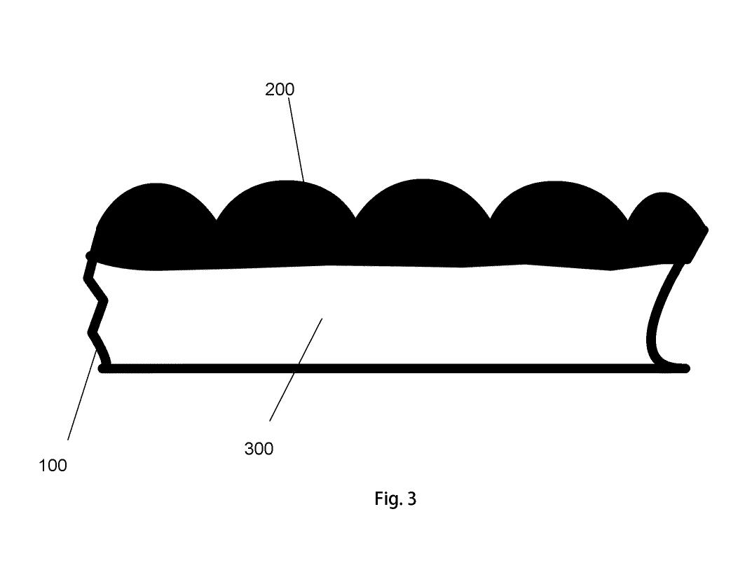

Now turning to FIG. 2, the cross section of garment assembly (100) is shown. In one implementation, the garment assembly 100 has an outer portion and an inner portion. The outer portion defines an outside area (200), which pertains to an inflatable compartment being substantially within the outer portion of the garment assembly. The inflatable compartment is made of elastic material that may be inflated when air fills up the inner portion. The inside area (300) pertains to a compartment being substantially within the inner portion of the garment assembly. The inside area disperses gas into the inflatable compartment when the protective device is activated. The invention does not depend on multiple separate inflatable chambers for coverage of the body areas to be protected. Such devices may not fully protect all the areas if one of the inflatable air chambers fails to inflate and fully expand. This is because the inflation and movement of any one air chamber to cover an area of the body is dependent upon the full inflation and movement of the adjacent chambers. This is particularly true for the uppermost and lowermost body areas protected by the device. At this point, as shown in FIG. 3, air pressure increases in the inflatable compartment, causing the elastic material to expand outward. Consequently, the body of the elderly man hidden within the inside area (300) of the garment assembly (100) may be protected with the expanded elastic material absorbing abundant of the impact resulting from the collision when hitting the ground.

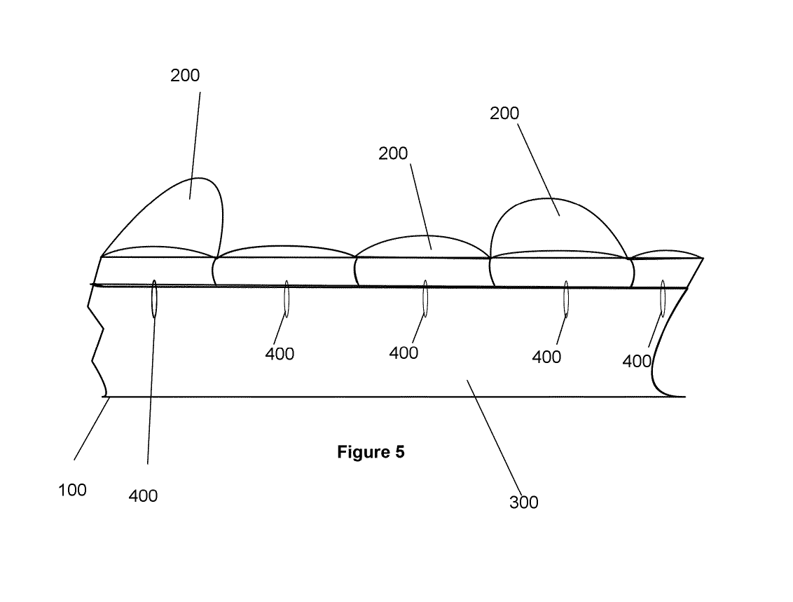

Different means to disperse gas from the inside area (300) to the outside area (200) may be employed. In one example, compressed gas may be stored in the inside area. When actuated, as a result of detecting a fall is about to occur, the compressed gas may be released into the outside area. In one further embodiment, the compressed gas, such as hydrogen gas, may be transferred through a valve (400), as shown in FIG. 5. The protective device is comprised of an airbag or airbags with portions folded into several layers contained within the device, which on inflation expand out of the protective device to cover the entire body area to be protected. Inflation and expansion are not hindered by internal attachments between the inside and outside portions. Alternatively, rather than supplying gas from inside area to the outside area through the valve, a plurality of chemical compound with liquid, such as water, may be mixed in the outside area that causes production of gas in a short duration to expand the elastic material on the outside area.

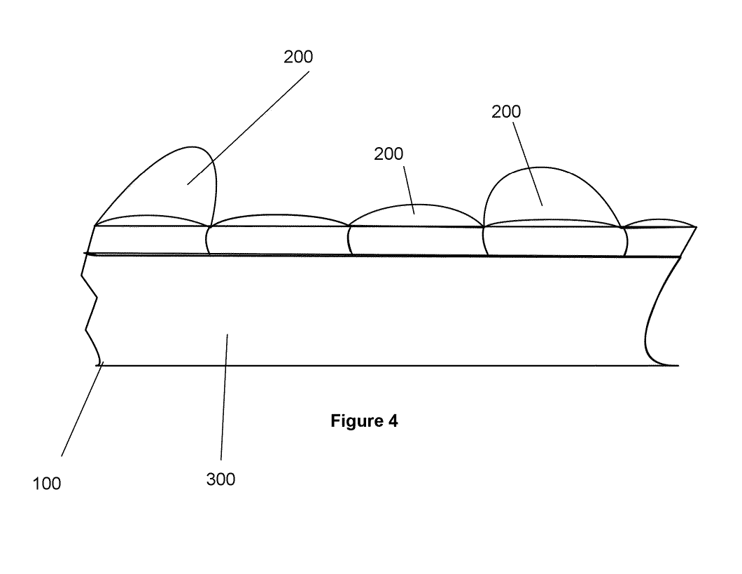

When it is detected that the elderly man is about to fall, the entire garment assembly may be inflated to protect the full protection. Advantageously, collision caused to different parts of the body of the falling person may be assured to be protected. However, there may be times where certain parts of the physical body require special attention more than others. As an example, the elderly man would prefer greater protection in a hip area that was injured previously. In which case, as shown in FIG. 4-5, each inflatable compartment of the insider area, which is composed of elastic material, may be made isolated from one another, so that a particular inflatable compartment may be adapted to inflate selectively to protect different parts of the elderly person.

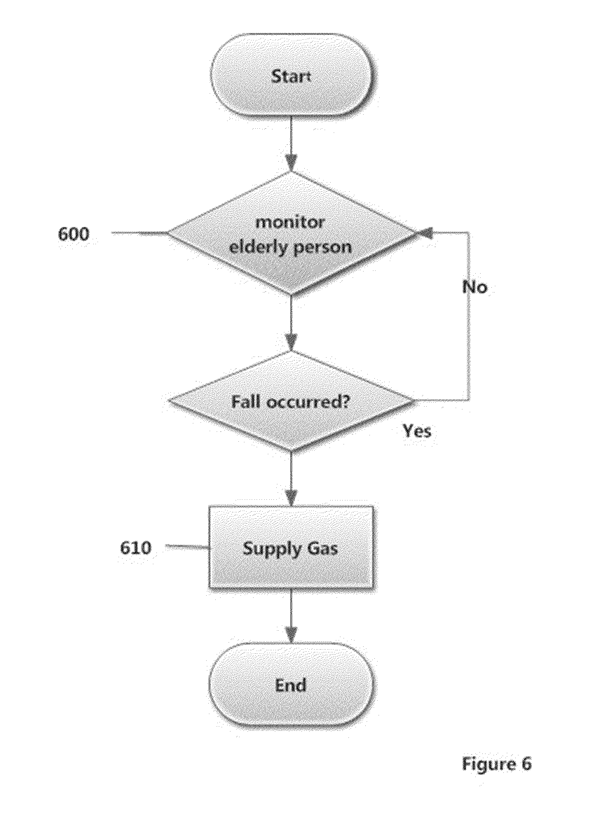

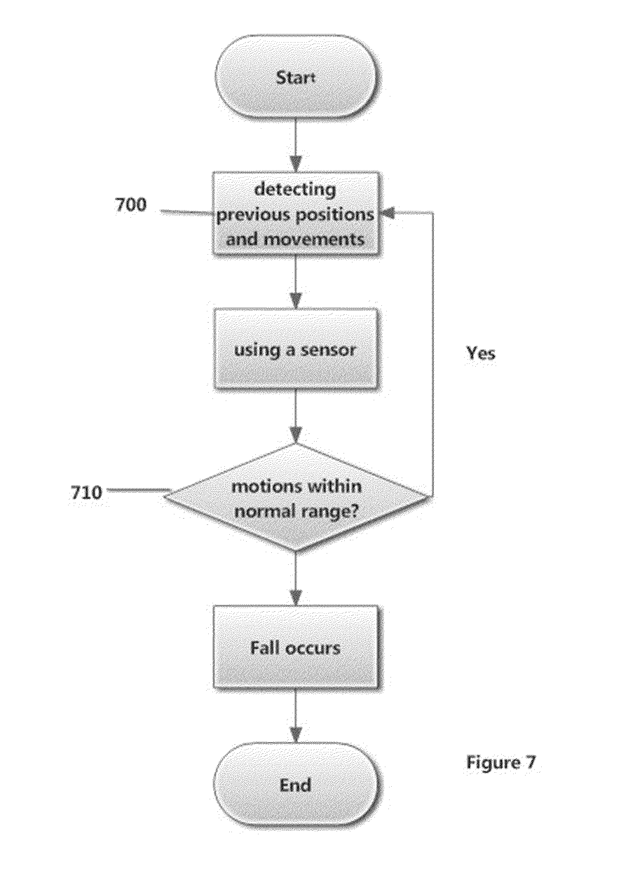

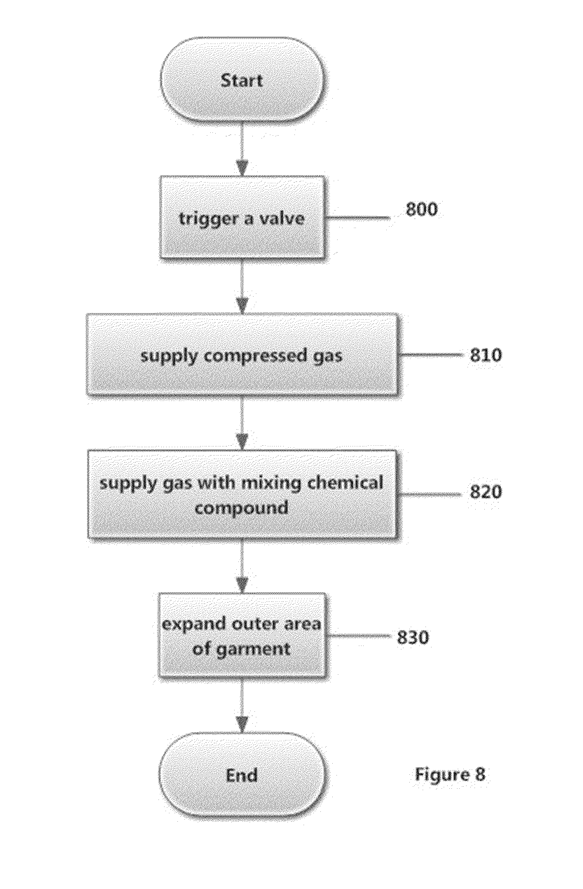

Turning now to FIGS. 6-8, methods are provided to prevent injuries caused to an elderly person. In one example, a method is provided to monitor whether a fall has occurred with respect to the elderly person (Step 600). When decided, gas is supplied from the inside area to the outside area (Step 610). Various means may be used to decide whether a fall has occurred with respect to the elderly person. For example, such means may include detecting based on the person's previous positions and movements by the sensor (Step 700), deciding whether the person's motions is within range of the person's previous normal positions and movements (Step 720), monitor parameters in speed of the fall and rate of change of speed of the fall, and monitoring positions including postural orientation. As regards to supplying the gas from the inside area to the outside area when the fall occurs, such method steps include triggering a valve connecting between the inside and outside area of the garment assembly (Step 800), supplying compressed gas from inside area to the outside area through the valve (Step 810), supplying gas from inside area to the outside area through the valve by means of mixing a plurality of chemical compound (Step 820), and expanding the outer area that reduces direct physical impact to the person (Step 830).

It is to be understood that the foregoing detailed description and accompanying drawings relate to a preferred illustrative embodiment of the invention. However, various changes and modifications may be made without departing from the spirit and scope of the invention. Regarding the sensor or sensors, as an example, any known form of angularly activated switch and any known type of accelerometer may be employed. Accordingly, the present invention is not limited to the specific arrangements as shown in the drawings and described in detail herein above. The exemplary materials, constructions and illustrations included in the preferred embodiment and this patent application should therefore not be construed to limit the scope of the present invention, which is defined by the appended claims.

While the disclosed invention has been taught with specific reference to the above embodiments, a person having ordinary skill in the art will recognize that changes may be made in form and detail without departing from the spirit and the scope of the invention. The described embodiments are to be considered in all respects only as illustrative and not restrictive. All changes that come within the meaning and range of equivalency of the claims are to be embraced within their scope. Combinations of any of the methods, systems, and devices described hereinabove are also contemplated and within the scope of the invention.

1. A protective device for preventing injuries to an elderly person, said device comprising:
a garment assembly having an outer portion and an inner portion, wherein the garment assembly is wearable by the elderly person;
an outside area pertaining to an inflatable compartment being substantially within the outer portion of the garment assembly, wherein the inflatable compartment is made of elastic material for protecting the elderly person during operation;
an inside area pertaining to a compartment being substantially within the inner portion of the garment assembly, wherein the inside area disperses gas into the inflatable compartment when the protective device is activated;
a sensor for detecting orientations, positions, and movements of the garment assembly;
a storage media for recording information received from the sensor; and
a processor configured to determine when a fall has occurred with respect to the elderly person and supply the gas from the inside area to the outside area when the fall occurs.

2. The protective device of claim 1, wherein the determination of when a fall has occurred with respect to the elderly person further comprises making a determination based on the previous positions and movements of the person.

3. The protective device of claim 2, wherein the detecting is made by the sensor.

4. The protective device of claim 3, wherein the determination of when a fall has occurred with respect to the elderly person further comprises deciding whether the person is within range of previous normal positions and movements of the person.

5. The protective device of claim 4, wherein a threshold value is predetermined to determine whether the person is within range of the person's previous normal positions and movements.

6. The protective device of claim 5, wherein it is determined that a fall has occurred when the person's current positions and movements has reached or exceeded a the threshold value.

7. The protective device of claim 6, wherein the determination of when a fall has occurred further comprises monitoring speed of the fall and rate of acceleration of the fall.

8. The protective device of claim 7, wherein the determination of when a fall has occurred further comprises monitoring positions including postural orientation.

9. The protective device of claim 8, wherein the supplying of gas from the inside area to the outside area further comprises triggering a valve connecting the inside and the outside area of the garment assembly.

10. The protective device of claim 9, wherein the supplying of gas from the inside area to the outside area further comprises supplying compressed gas from the inside area to the outside area through the valve.

11. The protective device of claim 9, wherein the supplying of gas from the inside area to the outside area further comprises supplying gas from inside area to the outside area through the valve by mixing a plurality of chemical compounds.

12. The protective device of claim 10, wherein the supplying of gas from the inside area to the outside area further comprises expanding the elastic material to inflate the outer area to reduce direct physical impact to the person.

13. A method to preventing injuries to a person, comprising:
deciding whether a fall has occurred with respect to the person; and
supplying gas from an inside area to the outside area of an inflatable garment assembly when the fall occurs.

14. The method of claim 13, wherein the step deciding whether a fall has occurred with respect to the a person further comprises:
detecting, using a sensor, based on previous positions and movements by the person;
deciding whether current motions of the person are within range of the previous normal positions and movements of the person;
monitoring speed parameters of the fall and rate of change of speed of the fall; and
monitoring positions including postural orientation.

15. The method of claim 14, wherein supplying the gas from the inside area to the outside area upon the fall occurring further comprises:
triggering a valve connecting the inside and the outside area of the garment assembly;
supplying compressed gas from the inside area to the outside area through the valve by mixing a plurality of chemical compound; and
expanding the outer area to reduce direct physical impact to the person.

16. A protective device for preventing injuries to an elderly person, said device comprising:
a garment assembly having an outer portion and an inner portion, wherein the garment assembly is wearable by the elderly person;
an outside area containing a plurality of inflatable compartments being substantially within the outer portion of the garment assembly, wherein each inflatable compartment, being made of elastic material, is isolated from one another, and is adapted to selectively inflate to protect different parts of the elderly person during operation;
an inside area pertaining to a compartment being substantially within the inner portion of the garment assembly, wherein the inside area disperses gas into each inflatable compartment selectively when the protective device is activated;
a sensor for detecting orientations, positions, and movements of the garment assembly;
a storage media for recording information received from the sensor; and
a processor configured to determine when a fall has occurred with respect to the elderly person and supply when the fall occurs, the gas from the inside area to at least one inflatable compartment of the outside area, wherein determining of the selection of the at least one inflatable compartment is based on the person's current movement, orientation, and position.

17. The protective device of claim 16, wherein the determination of when a fall has occurred with respect to the elderly person further comprises making a determination based on the previous positions and movements of the person.

18. The protective device of claim 17. wherein the detecting is made by the sensor.

19. The protective device of claim 18, wherein the determination of when a fall has occurred with respect to the elderly person further comprises deciding whether the elderly person is within range of the previous normal positions and movements of the elderly person.

20. The protective device of claim 19, wherein a threshold value is preconfigured and used to determine whether the elderly person is within range of the previous normal positions and movements of the elderly person.
